(19) United States
(12) Patent Application Publication
(10) Pub. No.: US 2008/0125871 A1
(43) Pub. Date: May 29, 2008

(54) Packaged Wrinkle-Filling Grafts and Method for Cosmetically Reducing the Appearance of Wrinkles

(76) Inventor: Mike Fard, Charlottesville, VA (US)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 11/941,512

(22) Filed: Nov. 16, 2007

Related U.S. Application Data
(60) Provisional application No. 60/866,180, filed on Nov. 16, 2006.

(51) Int. Cl. A61F 2/02

(52) U.S. Cl. 623/23.72; 604/11

(57) ABSTRACT

A fibrous, preferably crosslinked, graft material is pre-packaged in a sterile, water tight package to permit a surgeon with easy access to a safe, durable fill material that can significantly reduce the appearance of wrinkles on the skin surface. After performing a subcutaneous dissection under a wrinkle region to release the connections between the region and underlying planes of subcutaneous fat, muscle and the fibrotic bed, the surgeon fills a portion of the wrinkle region with the fibrous graft material to eliminate or reduce the appearance of wrinkles, folds and scars. The fibrous graft material may be housed in a tube that can be inserted under the skin, where the fibrous graft material is detachable and extrudable from the tube in situ at the wrinkle region.

### PRIORITY INFORMATION

This application claims priority to provisional application Ser. No. 60/866,180 filed Nov. 16, 2006, and the complete contents of this application is herein incorporated by reference.

### DESCRIPTION

### Background of the Invention

1. Field of the Invention

The present invention generally relates to methods and materials for cosmetically reducing the appearance of wrinkles, folds, or scars on a skin surface, such as the human face.

2. Background Description

To reduce the appearance of wrinkles, dermatologic plastic surgeons have traditionally utilized biologic or synthetic materials to generally augment tissue to remove wrinkles. For more complex facial wrinkles, such plastic surgeries involve the direct removal of redundant skin. However, both types of surgical procedures leave scars and can require a long recuperation time.

In recent years, the search for newer, simpler techniques with minimal invasive surgery have been pursued to address the appearance of wrinkled regions of skin (e.g., age-related wrinkles, etc.) For example, plastic surgeons have used fat harvested from other areas of a patient to remove the wrinkles on the patient's face. However this requires more time for the surgical procedure and poses increased risks to the patient due to the collection itself. The article “Wire Scalpel for Surgical Correction of Soft Tissue Contour Defects by Subcutaneous Dissection,”, Dermatol Surg. 26:2:February 2000, pgs 146-151, by M. Sulamandize et al., discuses subcutaneous dissection under the patient's wrinkle and involves releasing the connections between the wrinkle region and the underlying planes of subcutaneous fat, muscle, or the fibrotic bed. This procedure smoothes out and reduces the appearance of wrinkles. However, such a procedure may leave scars and may not smooth out all of the surface irregularities of the wrinkle region. In order to smooth out the remaining surface irregularities, some surgeons have used injectable fill products, such as Restylane® available from Q-med of Sweden. This product is a non-fibrous material that is injected at the wrinkle region to fill it out so as to reduce its appearance. Fill products such as Restylane® are composed of liquid or gel-like substances and do not have an internal structural support. Such fill products may be messy and inconvenient to use for the plastic surgeon. Moreover, the effects of these substances on the wrinkles general are not long lasting because they do not have internal structural integrity due to their non-fibrous character.

### SUMMARY OF THE INVENTION

An exemplary embodiment of the invention provides a simple, ready to use, prepackaged quantity of fibrous, and preferably crosslinked, graft material suitable for cosmetically reducing the appearance of wrinkles, folds or scars.

According to the invention, there is provided a package that is water tight, and sterile or sterilizable (e.g., foil, plastic, combinations thereof, etc.). The package includes at least one graft made of fibrous material that is present in sufficient quantity to fill a wrinkle, fold or scar after release. Preferably, the fibrous material includes constituents that are crosslinked or are crosslinkable upon application of radiant energy (e.g., UV, laser) or heat. The water tight character of the package retains moisture therein so that the graft will not dry out during storage prior to use. The fibrous material can be derived from porcine derma (e.g., Permacol-a available from Tissue Science Laboratories of England). Collagen derived from porcine derma, such as Permacol-a, is a rigid, but not hard, flexible crosslinked material that is acceptable for use in recontouring and repair of human tissue. However, many other fibrous materials that are safe for use in humans and animals can be employed. These materials may include animal collagen or wholly synthetic chemistries. The fibrous character of the material provides structural rigidity which will maintain its integrity over long periods of time and thus greatly prolong the effects of wrinkle reduction surgery. This structural integrity is enhanced when the fibers are crosslinked. The pre-packaged graft may be stick-shaped and may have a diameter of 1 mm, or 1 mm² area, and an approximate length of 5 cm. It is envisioned that grafts suitable for wrinkle reduction will generally be 1-3 mm in diameter or 1-4 mm in area, and be 1-20 cm in length. The shape and size of the graft can be altered in order to address different surgical objectives.

In practice, a surgeon performs subcutaneous dissection under a wrinkle region in the skin of a patient. This releases connections between skin wrinkles, folds or scars and the underlying planes of subcutaneous fat, muscle and the fibrotic bed. After dissection, at least a portion of the wrinkle region is filled with the fibrous graft material retrieved from the water tight, sterile package. In one embodiment, the filling step is performed by inserting a tube containing the graft material under the wrinkle region, then displacing the graft material from the tube and withdrawing the tube from the wrinkle region so as to deposit the material under the wrinkle region. A plunger may be used with the tube to assist in displacing the graft material. This procedure allows for the successful removal of the entire wrinkle without depressions or other sunken areas that can remain as a result of other known wrinkle-removing surgical procedures.

### BRIEF DESCRIPTION OF THE DRAWINGS

The foregoing and other objects, aspects and advantages will be better understood from the following detailed description of a preferred embodiment of the invention with reference to the drawings, in which:

FIGS. 1A and 1B show isometric views of the wrinkle-filling, fibrous graft material in specified shapes and sizes;

FIGS. 2A, 2B and 2C show exemplary packaging configurations which allow surgeons to use pre-packaged, wrinkle filling grafts when addressing appearance issues related to wrinkles, folds, and scars; and

FIG. 3 shows an exemplary tubular delivery system for depositing fibrous wrinkle filling grafts in situ at a surgical site.

### DETAILED DESCRIPTION OF THE PREFERRED EMBODIMENTS OF THE INVENTION

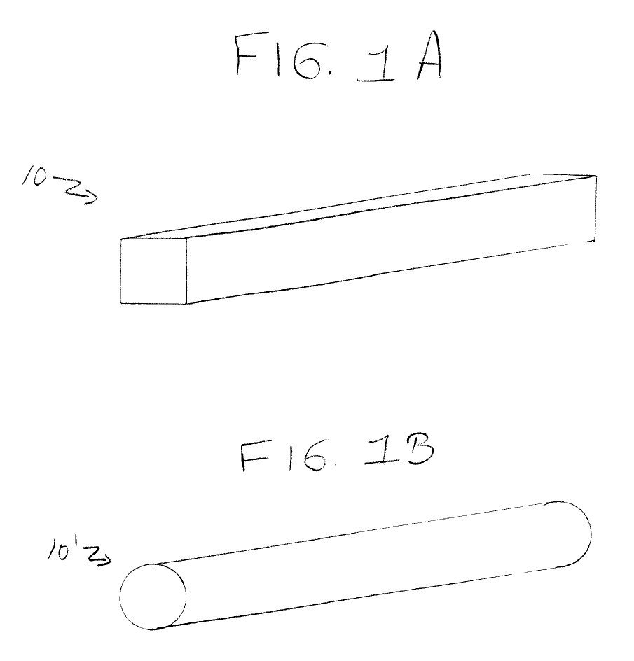

Referring now to the drawings, and more particularly to FIGS. 1A and 1B, there are shown different variations on a “stick shaped” configuration of the wrinkle-filling graft material. In practice, the surgeon will be able to retrieve a pre-shaped and pre-sized quantity of the graft material 10 to fill the area under a wrinkle region. For purposes of this invention, “wrinkle region” is defined as wrinkles, folds or scars on a skin surface of a patient. For purposes of this invention, the term “stick-shaped”can mean an elongated or extended box or tubular shape. FIGS. 1a and 1b respectively show a graft that is stick-shaped and has a diameter of 1 mm, or 1 mm² area, and an approximate length of 5 cm. In general, the graft may be 1-3 mm in diameter or 1-4 mm² in area and 1-20 cm in length. The wrinkle filling material may be in the form of an rectangular graft 10 or a tube-shaped graft 10′. However, it should be understood that different sizes and shapes (e.g., polygonal, triangular, etc.) may be used within the practice of this invention. Preferably, the graft 10 or 10′ is composed of collagen derived from porcine derma, or any other material that is fibrous in nature and which is compatible for use in humans and animals. In one embodiment, the fibers are crosslinked or are crosslinkable by application of UV, laser or heat energy to provide additional structural integrity. The graft 10 or 10′ can include animal collagen or be completely synthetic. Fibers in the graft provide structural rigidity. This rigidity can be enhanced by a crosslinked network. Hence, unlike prior gelatinous fill materials, the wrinkle filling graft material 10 or 10′ can retain its shape and structure after filling under the wrinkle for extended periods of time. Thus, the wrinkle filling graft 10 or 10′ should be soft and compatible for use in sensitive areas such as the human face, but have a fibrous character such that it can retain its function of masking the appearance of wrinkles over extended periods.

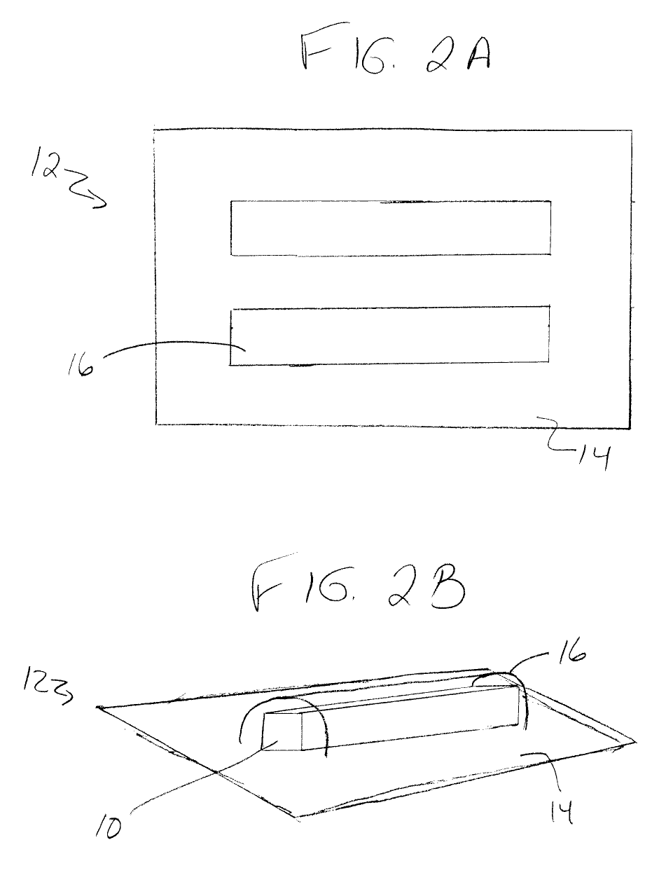

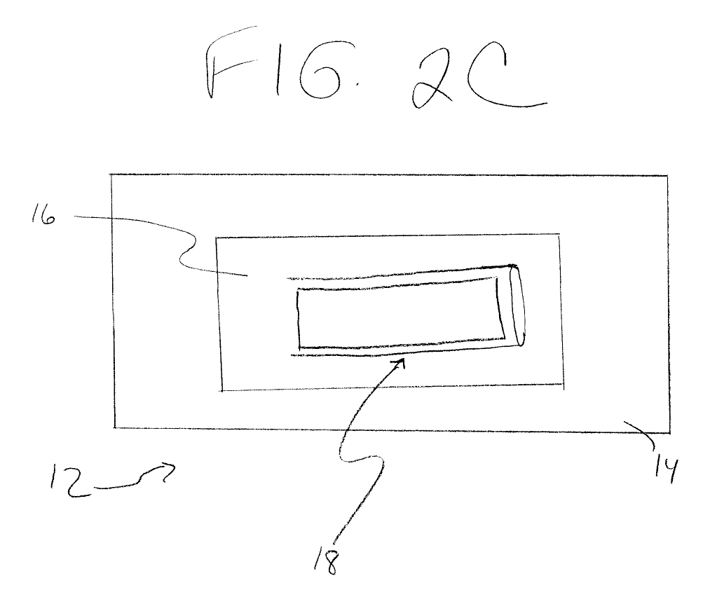

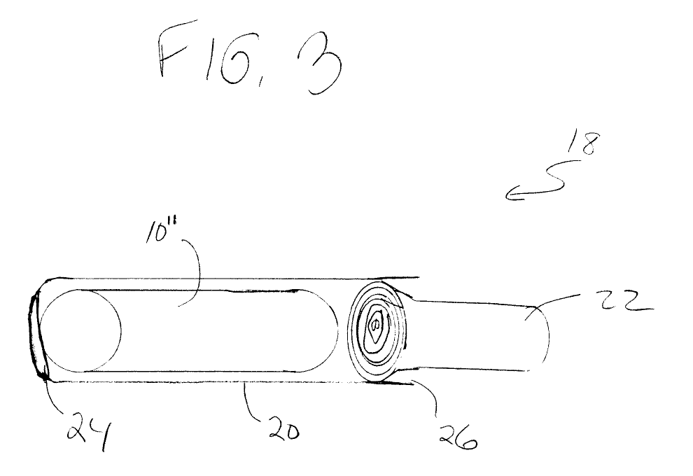

FIGS. 2A, 2B and 2C show exemplary packaging configurations for the wrinkle-filling graft material. The packaging 12 is made from a sterile or sterilizable material that is water tight, such as plastics, foil or combinations thereof, etc. The packaging 12 may be in the form of a flat sheet 14 with portion 16 that encloses the graft 10. The portion 16 may be a shrinkwrap-type enclosure that seals the graft 10 in a sterile environment. By ensuring that the graft 10 is sealed, moisture is retained and the graft 10 or 10′ does not dry out. This allows the graft 10 or 10′ to remain pliable during and after wrinkle-removing surgery.

FIG. 2C shows an alternative embodiment for the packaging which includes a tubular delivery system 18. The tubular delivery system 18, which can be cylindrical or polygonal, may be pre-filled with a graft 10 or 10′. A plunger (not shown) may also be included in the packaging with the tubular delivery system 18.

Although the packaging 12 in FIGS. 2A, 2B and 2C shows the graft 10 to be enclosed in a shrinkwrap-type enclosure on a flat sheet 14, other types of packaging (e.g., individually wrapped grafts in a box) can be used within the scope and purpose of the present invention. For example, the packaging can be a simple foil pouch similar to those used for hand-wipes.

A surgeon removes the graft 10 or 10′ from the packaging 12 after he or she has released the wrinkle by dissection. Because the graft 10 or 10′ is pre-shaped and sized with a and quantity of the wrinkle-filling fibrous material, no cutting or measuring of the graft is required. The surgeon then fills the portion of the wrinkle region with the graft 10 or 10′. Accordingly, wrinkle-removing procedures for surgeons is extremely simplified.

Referring now to FIG. 3, there is shown a tubular delivery system 18 which includes a hollow tube 20 with an entry 26, an exit 24, and a plunger 22. The graft 10″ is housed in a hollow tube 20. The tube 20 can be cylindrical or polygonal. The tube is preferably pre-packaged with the fibrous wrinkle filling graft material 10″ therein. The wrinkle filling graft material 10″ may be crosslinked or be crosslinkable by application of radiant or heat energy. Although the exit 24 is shown to be the approximate diameter of the tube 20, the exit 24 can be shaped into a tip of smaller diameter. The chief requirement is that the graft 10″ is displaceable from the tube by extrusion or other means using, for example, plunger 22. Upon making the subcutaneous dissection of the soft tissue at the wrinkle region, the surgeon inserts the tubular delivery system 18 into the wrinkle region such that the hollow tube 20 fills the region and the exit 24 is beneath the wrinkle. Once positioned, the surgeon displaces the graft 10″ in situ by, for example, pushing the plunger 22 and withdrawing the tube 20 so that the graft 10″ is pushed through the exit 24 to fill in the space under the wrinkle region.

The use of pre-packaged, fibrous (crosslinked) filling material, and preferably in situ extrusion under a released wrinkle, fold or scar prevents depressions or other sunken areas from occurring and ensures complete removed of the wrinkle over an extended period of time.

While the invention has been described in terms of its preferred embodiment, those skilled in the art will recognize that the invention can be practiced with modification within the spirit and scope of the appended claims.

Having thus described my invention, what I claim as new and desire to secure by Letters Patent is as follows:

1. A method for cosmetically reducing the appearance of wrinkles, folds or scars on a skin surface of a patient, comprising the steps of:
performing subcutaneous dissection under a wrinkle region in the skin of a patient, said performing subcutaneous dissection step releasing connections between skin wrinkles, folds or scars, and underlying planes of subcutaneous fat, muscle, and fibrotic bed; and
filling at least a portion of said wrinkle region after dissection with a fibrous graft material.

2. The method of claim 1 wherein said fibrous graft material includes collagen derived from porcine derma.

3. The method of claim 1 wherein said fibrous graft material is stick shaped.

4. The method of claim 1 further comprising the step of crosslinking at least a portion of said fibrous graft material.

5. The method of claim 1 wherein said filling step includes the steps of:
inserting a tube containing said fibrous graft material under said wrinkle region; and
displacing said fibrous graft material from said tube and withdrawing said tube from under said wrinkle region so as to deposit said fibrous, crosslinked graft material under said wrinkle region.

6. Packaged wrinkle filling graft for cosmetically reducing the appearance of wrinkles, folds or scars on a skin surface of a patient, comprising:
a fibrous graft material of a specified size and shape; and
a sterile, water tight package containing said fibrous graft material.

7. The packaged wrinkle filling graft of claim 6 wherein said fibrous graft material is synthetic.

8. The packaged wrinkle filling graft of claim 6 wherein said fibrous graft material includes collagen derived from porcine derma.

9. The packaged wrinkle filling graft of claim 6 wherein said fibrous graft material is stick shaped.

10. The packaged wrinkle filling graft of claim 9 wherein said specified size of said fibrous graft material is approximately 1 mm in diameter or 1 mm² in area and approximately 5 cm in length.

11. The packaged wrinkle filling graft of claim 9 wherein said specified size of said fibrous graft material is 1-3 mm in diameter or 1-4 mm² in area and 1-20 cm in length.

12. The packaged wrinkle filling graft of claim 6 wherein said fibrous graft material is crosslinked.

13. The packaged wrinkle filling graft of claim 6 wherein said sterile, water tight package is made of plastic.

14. The packaged wrinkle filling graft of claim 6 wherein said sterile, water tight package is made of foil.

15. The packaged wrinkle filling graft of claim 6 further comprising a tube for releasably holding said fibrous graft material such that said fibrous, crosslinked graft material can be extruded or otherwise displaced from said tube.
